(19) United States
(12) Patent Application Publication
(10) Pub. No.: US 2026/0273281 A1
(43) Pub. Date: Sep. 17, 2026

(54) ARTEFACT VISUALIZATION TOOL

(71) Applicant: Cochlear Limited, Macquarie University, New South Wales (AU)

(72) Inventors: Guy FIERENS, Mortsel (BE); Matthias GRAMSS, Niederneisen (DE)

(21) Appl. No.: 19/473,390

(22) PCT Filed: Apr. 5, 2024
(86) PCT No.: PCT/IB2024/053359

§ 371 Date: Oct. 7, 2025

Related U.S. Application Data
(60) Provisional application No. 63/495,572, filed on Apr. 12, 2023.

(51) Int. Cl. A61N 1/36; A61B 34/10; A61B 90/00; G01R 33/56

(52) U.S. Cl. CPC A61N 1/36039; A61B 34/10; G01R 33/5608; A61B 2034/104; A61B 2034/105; A61B 2090/374

(57) ABSTRACT

Presented herein are techniques for implant-induced artefact visualization using pre-operative MRI imaging data. Operations include obtaining pre-operative magnetic resonance imaging (MRI) data, and obtaining a three-dimensional (3D) artefact model 2024/213975 for an implant of a recipient based on an implant type. The operations further include overlaying the 3D artefact model of the implant on the pre-operative MRI data based on a given implant location, and displaying the pre-operative MRI data with the 3D artefact model of the implant overlaid thereon so as to visualize an implant-induced image artefact for the recipient according to the given implant location. WO

### BACKGROUND

### Field of the Invention

The present invention relates generally to techniques for implant-induced artefact visualization.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: obtaining pre-operative magnetic resonance imaging (MRI) data; obtaining a three-dimensional (3D) artefact model for an implant of a recipient based on an implant type; and displaying the pre-operative MRI data with the 3D artefact model of the implant overlaid thereon so as to visualize an implant-induced image artefact for the recipient according to the given implant location.

In another aspect, one or more non-transitory computer readable storage media are provided. The non-transitory computer readable storage media comprise instructions that, when executed by a processor, cause the processor to: obtain imaging data associated with an anatomical region of a recipient of an implantable component prior to implantation of the implantable component in the anatomical region; obtain a three-dimensional (3D) artefact model for the implantable component; determine a selected implantable location for the implantable component in the anatomical region; and display the imaging data in combination with the 3D artefact model for the selected implantable location.

In another aspect, a system is provided. The system comprises: a display screen; a memory storing computer-readable instructions; and at least one processor operably coupled to the display screen and the memory, wherein the at least one processor is configured to execute the computer-readable instructions to: obtain pre-operative imaging data associated with a recipient; obtain an artefact model associated with an implant configured to be implanted in the recipient; and display, on the display screen, the artefact model associated with the implant overlaid on pre-operative imaging data according to a given implant location.

In another aspect, a method is provided. The method comprises: obtaining pre-operative medical imaging data of a recipient of an implant; generating a three-dimensional (3D) model for the implant based on a selected implant type; and displaying the pre-operative medical imaging data, along with a 3D artefact volume based on a given implant location, so as to visualize an implant-induced image artefact for the recipient.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing device with which aspects of the techniques presented herein can be implemented;

FIG. 2 is an example workflow process for magnetic resonance imaging (MRI) artefact visualization using pre-operative imaging data, according to an example embodiment;

FIG. 3A shows a GUI screen that is displayed when the “select recipient” tab is selected, according to an example embodiment;

FIG. 3B shows a GUI screen that is displayed when the “select implant” tab is selected, according to an example embodiment;

FIG. 3C shows a GUI screen that is displayed when the “implant location” tab is selected, according to an example embodiment;

FIG. 3D shows a GUI screen that is displayed when the “annotated data” tab is selected, according to an example embodiment;

FIG. 3E shows a GUI screen that is displayed when the annotated data tab is selected, according to an example embodiment;

FIG. 4 is a flowchart of a method for artefact visualization using pre-operative imaging data, according to an example embodiment;

FIG. 5 is a flowchart of a method for artefact visualization using pre-operative imaging data, according to another example embodiment; and FIG. 6 is a schematic diagram illustrating a vestibular stimulator system with which aspects of the techniques presented herein can be implemented.

### DETAILED DESCRIPTION

### Overview

Presented herein are techniques for implant-induced artefact visualization using pre-operative imaging data, such as Magnetic resonance imaging (MRI) data. Operations include obtaining pre-operative imaging data (e.g., MRI data), and obtaining a three-dimensional (3D) artefact model for an implant/implantable component of a recipient based on an implant type. The operations further include overlaying the 3D artefact model of the implant on the pre-operative imaging data based on a given implant location, and displaying the pre-operative imaging data with the 3D artefact model of the implant overlaid thereon so as to visualize an implant-induced image artefact for the recipient according to the given implant location. As used herein, the term “recipient” generally refers to an individual who has received, or is a candidate to receive, an implant/implantable component of a medical device.

There are a number of different types of devices in/with which embodiments of the present invention can be implemented. Merely for ease of description, the techniques presented herein are primarily described with reference to a specific device in the form of a cochlear implant system. However, it is to be appreciated that the techniques presented herein can also be partially or fully implemented by any of a number of different types of devices, including consumer electronic device (e.g., mobile phones), wearable devices (e.g., smartwatches), hearing devices, implantable medical devices, wearable devices, etc. consumer electronic devices, wearable devices (e.g., smart watches, etc.), etc. As used herein, the term “hearing device” is to be broadly construed as any device that delivers sound signals to a user in any form, including in the form of acoustical stimulation, mechanical stimulation, electrical stimulation, etc. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems. combinations or variations thereof, etc.) or a device for use by a person with normal hearing (e.g., consumer devices that provide audio streaming, consumer headphones, earphones, and other listening devices). In other examples, the techniques presented herein can be implemented by, or used in conjunction with, various implantable medical devices, such as vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

### Example System and Components

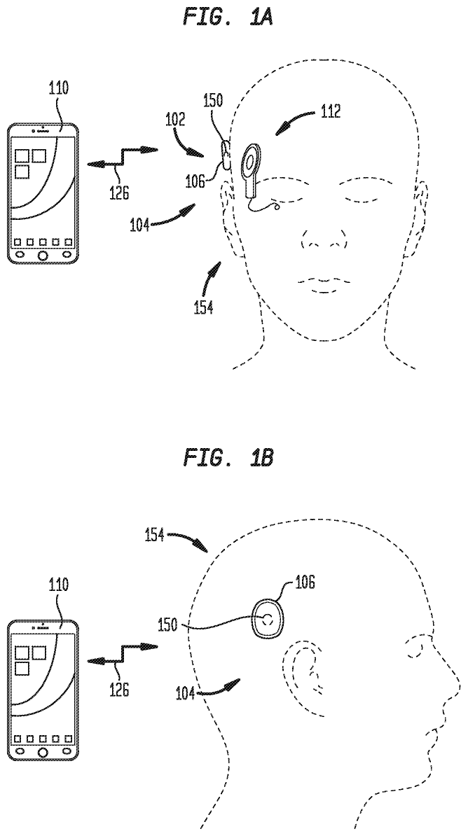

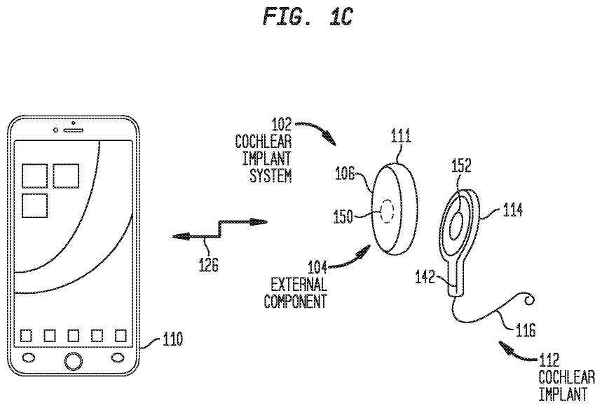

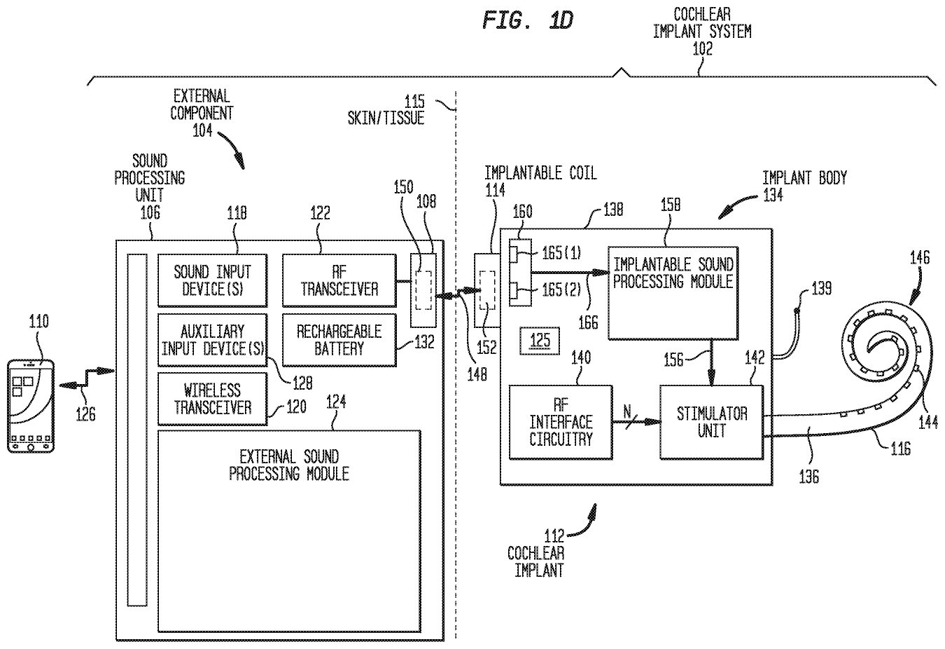

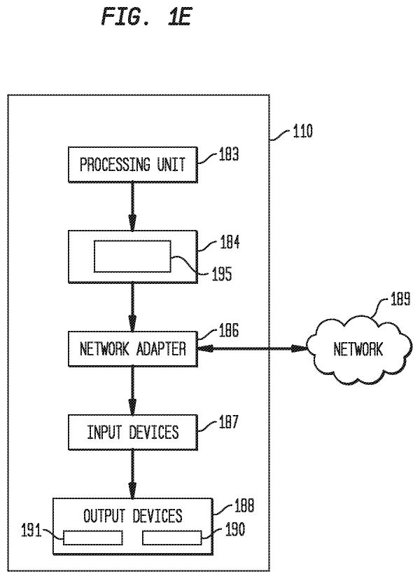

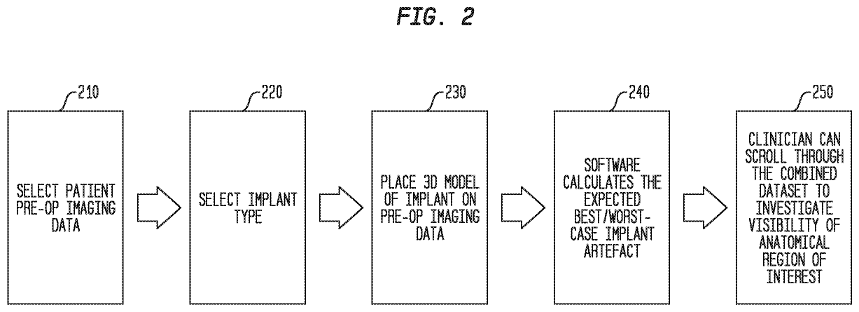

FIGS. 1A-1D illustrates an example cochlear implant system 102 with which aspects of the techniques presented herein can be implemented. The cochlear implant system 102 comprises an external component 104 that is configured to be directly or indirectly attached to the body of the user, and an internal/implantable component 112 that is configured to be implanted in or worn on the head of the user. In the examples of FIGS. 1A-1D, the implantable component 112 is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

In the examples of FIGS. 1A-1D, the external component 104 comprises a sound processing unit 106, an external coil 108, and generally, a magnet fixed relative to the external coil 108. The cochlear implant 112 includes an implantable coil 114, an implant body 134, and an elongate stimulating assembly 116 configured to be implanted in the user's cochlea. In one example, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the user's head 154 (e.g., includes an integrated external magnet 150 configured to be magnetically coupled to an internal/implantable magnet 152 in the implantable component 112). The OTE sound processing unit 106 also includes an integrated external (headpiece) coil 108 (the external coil 108) that is configured to be inductively coupled to the implantable coil 114.

It is to be appreciated that the OTE sound processing unit 106 is merely illustrative of the external devices that could operate with implantable component 112. For example, in alternative examples, the external component 104 can comprise a behind-the-ear (BTE) sound processing unit configured to be attached to, and worn adjacent to, the recipient's ear. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

Although the cochlear implant system 102 includes the sound processing unit 106 and the cochlear implant 112, as described below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110, which is shown in greater detail in FIG. IE, is a computing device, such as a personal computer (e.g., laptop, desktop, tablet), a mobile phone (e.g., smartphone), remote control unit, etc. The external device 110 and the cochlear implant system 102 (e.g., sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-1D, the sound processing unit 106 of the external component 104 also comprises one or more input devices configured to capture and/or receive input signals (e.g., sound or data signals) at the sound processing unit 106. The one or more input devices include, for example, one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a short-range wireless transmitter/receiver (wireless transceiver) 120 (e.g., for communication with the external device 110), each located in, on or near the sound processing unit 106. However, it is to be appreciated that one or more input devices can include additional types of input devices and/or less input devices (e.g., the short-range wireless transceiver 120 and/or one or more auxiliary input devices 128 could be omitted).

The sound processing unit 106 also comprises the external coil 108, a charging coil 130, a closely-coupled radio frequency transmitter/receiver (RF transceiver) 122, at least one rechargeable battery 132, and an external sound processing module 124. The external sound processing module 124 can be configured to perform a number of operations and can be formed by one or more processors (e.g., one or more Digital Signal Processors (DSPs), one or more uC cores, etc.), firmware, software, etc. arranged to perform operations described herein. That is, the external sound processing module 124 can be implemented as firmware elements, partially or fully implemented with digital logic gates in one or more application-specific integrated circuits (ASICs), partially or fully in software, etc.

Returning to the example of FIGS. 1A-1D, the implantable component 112 comprises an implant body (main module) 134, a lead region 136, and the intra-cochlear stimulating assembly 116, all configured to be implanted under the skin (tissue) 115 of the user. The implant body 134 generally comprises a hermetically-sealed housing 138 that includes, in certain examples, at least one power source 125 (e.g., one or more batteries, one or more capacitors, etc.), in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact array (electrode array) 146 for delivery of electrical stimulation (current) to the recipient's cochlea. Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108 and the internal/implantable magnet 152 is fixed relative to the implantable coil 114. The external magnet 150 and the internal/implantable magnet 152 fixed relative to the external coil 108 and the internal/implantable coil 114, respectively, facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive, and/or inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to process the received input audio signals (received at one or more of the input devices, such as sound input devices 118 and/or auxiliary input devices 128), and convert the received input audio signals into output control signals for use in stimulating a first ear of a recipient or user (i.e., the external sound processing module 124 is configured to perform sound processing on input signals received at the sound processing unit 106). Stated differently, the one or more processors (e.g., processing element(s) implementing firmware, software, etc.) in the external sound processing module 124 are configured to execute sound processing logic in memory to convert the received input audio signals into output control signals (stimulation signals) that represent electrical stimulation for delivery to the recipient.

As noted, FIG. 1D illustrates an embodiment in which the external sound processing module 124 in the sound processing unit 106 generates the output control signals. In an alternative embodiment, the sound processing unit 106 can send less processed information (e.g., audio data) to the implantable component 112 and the sound processing operations (e.g., conversion of input sounds to output control signals 156) can be performed by a processor within the implantable component 112.

In FIG. 1D, according to an example embodiment, output control signals (stimulation signals) are provided to the RF transceiver 122, which transcutaneously transfers the output control signals (e.g., in an encoded manner) to the implantable component 112 via external coil 108 and implantable coil 114. That is, the output control signals (stimulation signals) are received at the RF interface circuitry 140 via implantable coil 114 and provided to the stimulator unit 142. The stimulator unit 142 is configured to utilize the output control signals to generate electrical stimulation signals (e.g., current signals) for delivery to the user's cochlea via one or more of the stimulating contacts (electrodes) 144. In this way, cochlear implant system 102 electrically stimulates the user's auditory nerve cells, bypassing absent or defective hair cells that normally transduce acoustic vibrations into neural activity, in a manner that causes the recipient to perceive one or more components of the input audio signals (the received sound signals).

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, an example embodiment of the cochlear implant 112 can include a plurality of implantable sound sensors 165(1), 165(2) that collectively form a sensor array 160, and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 165(1), 165(2) of the sensor array 160 are configured to detect/capture input sound signals 166 (e.g., acoustic sound signals, vibrations, etc.), which are provided to the implantable sound processing module 158. The implantable sound processing module 158 is configured to convert received input sound signals 166 (received at one or more of the implantable sound sensors 165(1), 165(2)) into output control signals 156 for use in stimulating the first ear of a recipient or user (i.e., the implantable sound processing module 158 is configured to perform sound processing operations). Stated differently, the one or more processors (e.g., processing element(s) implementing firmware, software, etc.) in implantable sound processing module 158 are configured to execute sound processing logic in memory to convert the received input sound signals 166 into output control signals 156 that are provided to the stimulator unit 142. The stimulator unit 142 is configured to utilize the output control signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 165(1), 165(2) of sensor array 160 in generating stimulation signals for delivery to the user.

FIG. 1E is a block diagram illustrating one example arrangement for an external computing device 110 configured to perform one or more operations in accordance with certain embodiments presented herein. As shown in FIG. 1E, in its most basic configuration, the external computing device 110 includes at least one processing unit 183 and a memory 184. The processing unit 183 includes one or more hardware or software processors (e.g., Central Processing Units) that can obtain and execute instructions. The processing unit 183 can communicate with and control the performance of other components of the external computing device 110. The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 184 can include transitory memory or non-transitory memory. The memory 184 can also include one or more removable or non-removable storage devices. In examples, the memory 184 can include random access memory (RAM), read only memory (ROM), EEPROM (Electronically-Erasable Programmable Read-Only Memory), flash memory, optical disc storage, magnetic storage, solid state storage, or any other memory media usable to store information for later access. By way of example, and not limitation, the memory 184 can include wired media such as a wired network or direct-wired connection, and wireless media such as acoustic, RF, infrared and other wireless media or combinations thereof. In certain embodiments, the memory 184 comprises logic 195 that, when executed, enables the processing unit 183 to perform aspects of the techniques presented (e.g., provides a software tool for implant-induced artefact visualization using pre-operative imaging data).

In the illustrated example of FIG. 1E, the external computing device 110 further includes a network adapter 186, one or more input devices 187, and one or more output devices 188. The external computing device 110 can include other components, such as a system bus, component interfaces, a graphics system, a power source (e.g., a battery), among other components. The network adapter 186 is a component of the external computing device 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols. The one or more input devices 187 are devices over which the external computing device 110 receives input from a user. The one or more input devices 187 can include physically-actuatable user-interface elements (e.g., buttons, switches, or dials), a keypad, keyboard, mouse, touchscreen, and voice input devices, among other input devices that can accept user input. The one or more output devices 188 are devices by which the computing device 110 is able to provide output to a user. The output devices 188 can include, for example, a display 190 (e.g., a liquid crystal display (LCD)) and one or more speakers 191, among other output devices for presentation of visual or audible information to the recipient, a clinician, an audiologist, or other user (e.g., displays GUI screens for implant-induced artefact visualization using pre-operative imaging data).

It is to be appreciated that the arrangement for the external computing device 110 shown in FIG. 1E is merely illustrative and that aspects of the techniques presented herein can be implemented at a number of different types of systems/devices including any combination of hardware, software, and/or firmware configured to perform the functions described herein. For example, the external computing device 110 can be a personal computer (e.g., a desktop or laptop computer), a hand-held device (e.g., a tablet computer), a mobile device (e.g., a smartphone), a surgical system, and/or any other electronic device having the capabilities to perform the associated operations described elsewhere herein.

### Example Embodiments

As noted, techniques are provided herein where implant artefact data (e.g., an implant magnet induced artefact image) associated with an implantable component (implant) is overlaid with a pre-operative imaging data of a recipient (e.g., imaging data of a recipient). Overlaying the implant artefact data on the pre-operative imaging data enables a determination of which parts of the recipient's anatomy is likely to be visible in the presence of the implant (e.g., what portions of the recipient's anatomy is likely to be visible when the implant is subsequently implanted in the recipient). The techniques presented herein can be used in a pre-operative plan for a new implant recipient to determine, for example, where to place the implant in the recipient. The techniques can also be used post-operatively to assist in determining whether a magnet of the implant needs to be removed prior to performing medical imaging on the recipient.

Current guidelines for active implantable medical devices (AIMD) describe the “size” of implant-induced image artefacts using a single metric, which is typically the artefact radius in an axial plane as measured from the implant center (or artefact diameter). As used herein, the term “implant-induced image artefact” refers to the affect that the implant has, or is likely to have, on a medical image performed in the presence of the implant (e.g., while the implant is implanted in the recipient). As used herein, an implant-induced MRI artefact is implant-induced image artefact with reference to a Magnetic Resonance Imaging (MRI) scan.

Using one single value to describe an implant-induced artefact, such as an implant-induced MRI artefact, is a fairly rudimentary approach, with the sole purpose of managing the expectations of the clinicians for post-operative imaging. A drawback of a single value is that it fails to account for the typically somewhat irregular shape of the artefact (e.g., more pear-shaped rather than circular). That is a single radius measurement does not accurately represent a typical artefact, which are complex three-dimensional (3D) volumes.

The solution of the present disclosure provides an alternative to the state-of-the-art described above, and is particularly useful in complex cases where a more elaborate approach (than using a one dimensional metric, such as a radius) is required. A concept for a tool (e.g., software tool) is proposed where the artefact “volume” (i.e., a three dimensional metric) for a certain implantable medical device can be overlaid (or combined/merged) with the pre-operative imaging dataset of a recipient. The techniques presented herein provide more standardized artefact dimensioning to create a degree of uniformity. By automating accurate artefact measurements in three dimensions and allowing visualization of an implant-induced image artefact in 3D, the techniques presented can provide more realistic artefact dimensioning than existing solutions. In some example embodiments, the artefact volume could be provided by the device manufacturer, or can be calculated using a robust image artefact measurement algorithm. In the case of a pediatric recipient that is still growing, an anonymized dataset can be used for an appropriate demographic comparison.

One technique to quantify an artefact includes measuring the extent of the artefact using, for example, a software tool that allows visualizing the images. Another method involves outlining the artefact using a thresholding technique. In this example, an image with an artefact is overlaid (combined, merged) with an image without an artefact, and both are subtracted to allow the clinician/user to look at the pixels that have changed X % in intensity between the two images. Based on this, the pixels can be classified as either ‘artefact’ or ‘no-artefact.’ A mask is then created, which can be used to more accurately measure the artefact extent (similar to the aforementioned manual measurement technique). According to a further technique, the artefact volume can be calculated (in mm³) based on the voxel size, and an algorithm can be used to report the actual size of the artefact in an understandable way for the clinician/user.

Generally, example embodiments described herein provide a system and methods for generating and displaying 3D models that can be overlaid in common Digital Imaging and Communications in Medicine (DICOM) viewers (e.g., general purpose software like 3D slicer, or cochlear-specific software). Presenting a 3D model of an MRI artefact shape to clinicians as an overlay on pre-operative imaging data (a pre-implantation scan) enables a determination to be made regarding whether a region of interest (ROI) can be visualized using MRI, for example.

The proposed techniques presented herein, e.g., embodied as software tool, enable a clinician to have a detailed look around the site of implantation for a “virtual” MRI scan. In other words, the techniques presented herein enable clinicians to overlay expected artefact sizes for a given implant position in a “virtual recipient.” In turn, this enables assessment on a more personal level regarding what parts of the anatomy can be reliably visualized. For example, a best-case and worst-case artefact volume (or a defined range of artefact volume) can be provided by the manufacturer, or can be calculated using the image artefact measurement algorithm.

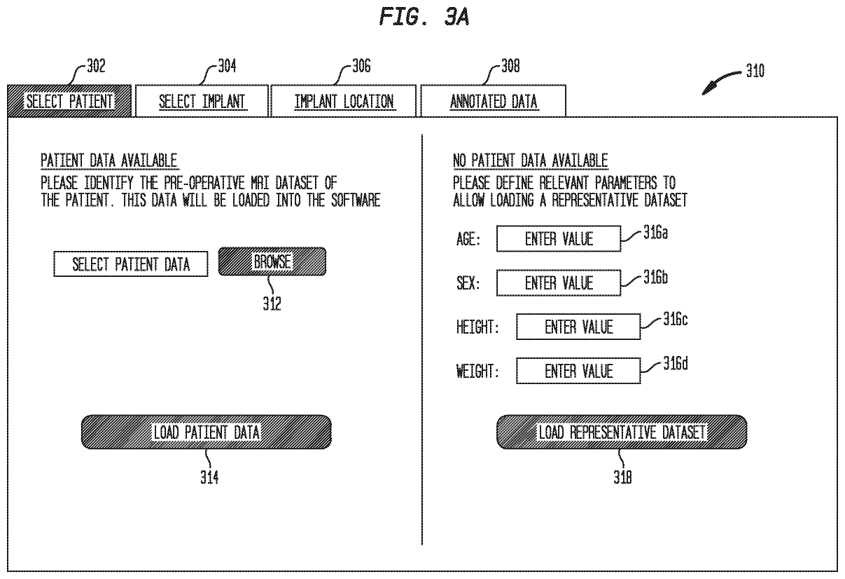

A practical use case, in which recipient imaging data can be overlaid with known image artefact volumes, will now be described with reference to FIGS. 2, 3A-3E, and 4. Assume that a clinician wants to see for his recipient (or a similarly sized recipient, if there is no pre-op imaging data available) how the presence of the implant affects this specific case. The clinician can then import the 3D artefact model in the DICOM viewer together with the recipient dataset (or the dataset for the similarly sized recipient), and overlay the 3D artefact model on the site where the implant is located. This enables the clinician to assess, in this specific case, what the impact of the implant would be on the diagnostic capabilities of MRI in this specific recipient. This helps in setting the expectations, and also in pre-operative planning in case a certain area of the head needs to be preserved.

FIG. 2 is a workflow process 200 for MRI artefact visualization using pre-operative imaging data, according to an example embodiment.

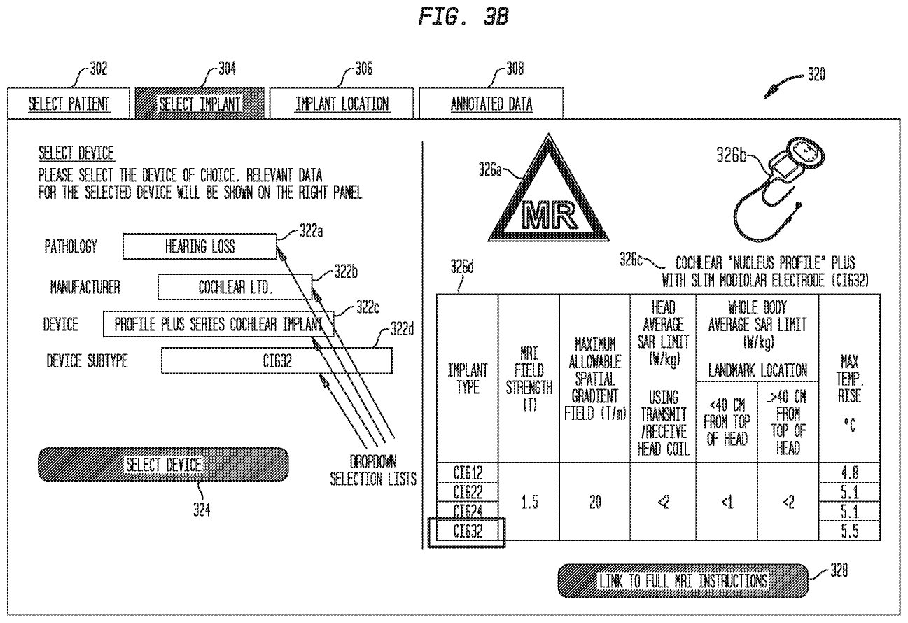

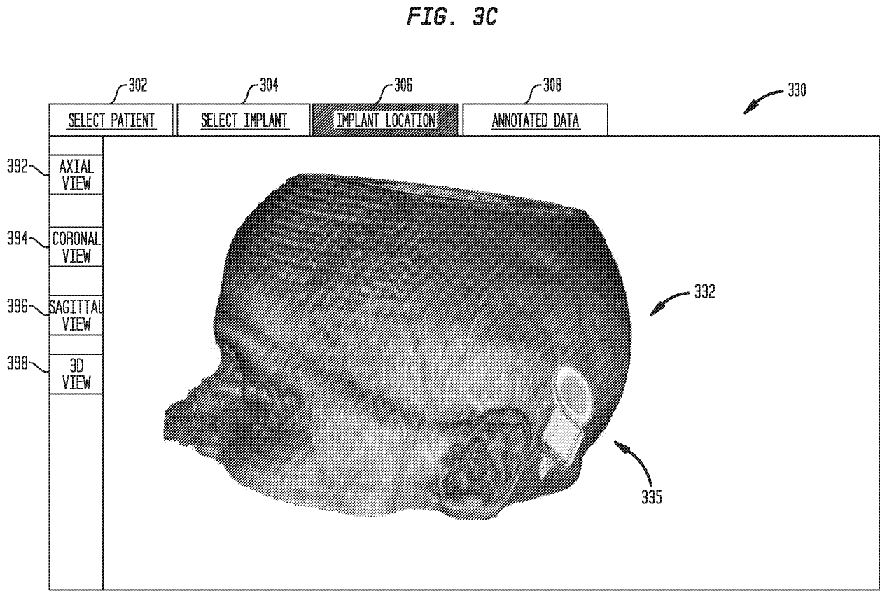

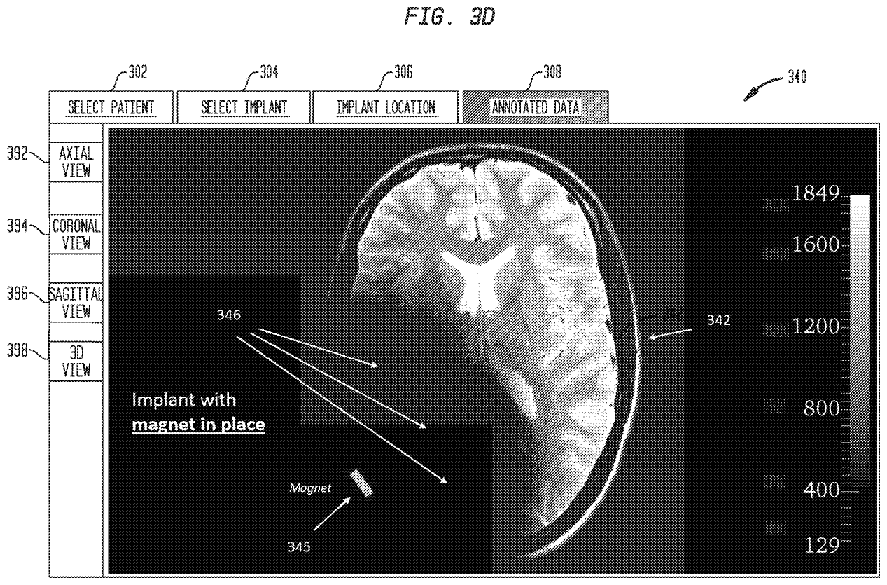

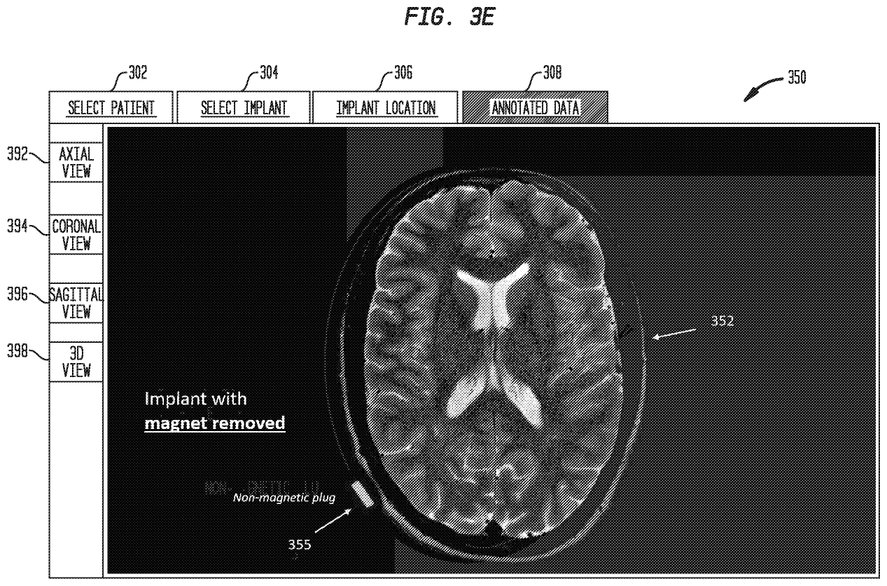

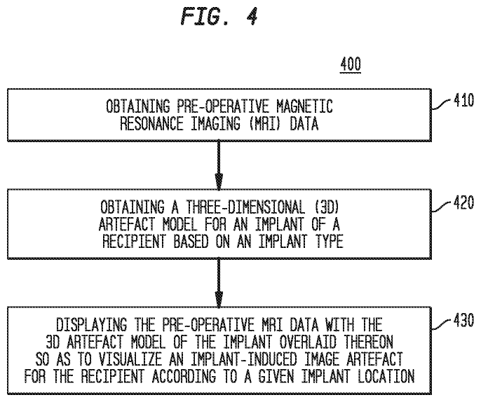

The workflow process 200 begins at operation 210, where a user selects recipient pre-operative imaging data (MRI). An example implementation for this operation is shown in FIG. 3A. The user then selects an implant type, at operation 220. An example implementation for this operation is shown in FIG. 3B.

At operation 230, the techniques presented herein are used to place a 3D model of the implant (overlay) on the pre-operative imaging data. An example implementation for this operation is shown in FIGS. 3C and 3D. At operation 240, the techniques presented herein calculate an expected best-case implant artefact volume and an expected worst-case implant artefact volume. In some embodiments, the techniques are embodied as a software tool for quantifying an artefact described above, for example.

The techniques presented herein cause display of a combined dataset at operation 250, which enables the user to scroll through the combined dataset to investigate visibility (or lack thereof) of one or more anatomical regions of interest (ROIs). An example implementation for this operation is shown in FIGS. 3D and 3E.

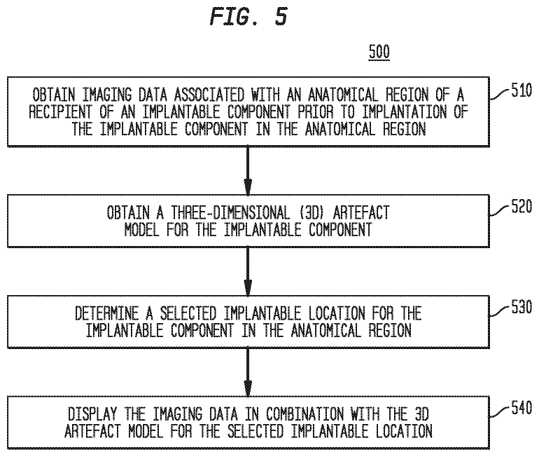

FIGS. 3A-3E show various graphical user interface (GUI) screens of an example software tool, which can be used to implement steps of the workflow process 200 of FIG. 2 and a method 400 described below with reference to FIG. 4. As shown in FIGS. 3A-3E, the GUI 300 of the software tool has a row of tabs along the top of the window, including a “select recipient” tab 302, a “select implant” tab 304, an “implant location” tab 306, and an “annotated data” tab 308.

FIG. 3A shows a GUI screen 310 that is displayed by the software tool when the “select recipient” tab 302 is selected, according to an example embodiment. If recipient data is available, the user can identify the pre-operative MRI dataset of the recipient (e.g., by selecting the “browse” button 312), and load this recipient data into the software (e.g., by selecting the “load recipient data” button 314), as shown on the left side of FIG. 3A. If recipient data is not available (i.e., there is no pre-operative MRI for the recipient), the user can define relevant parameters (e.g., enter values for age, sex, height, weight in the recipient parameter fields 316a, 316b, 316c, and/or 316d, respectively), and load a representative dataset into the software (e.g., by selecting the load representative dataset button 318), as shown on the right side of FIG. 3A.

FIG. 3B shows a GUI screen 320 that is displayed by the software tool when the “select implant” tab 304 is selected, according to an example embodiment. As shown on the left side of FIG. 3B, the user can select a device of choice. For example, the user can enter, or select from corresponding dropdown lists, a pathology 322a, a manufacturer 322b, a device 322c, and/or a device subtype 322d, respectively, non-limiting examples of which are shown in corresponding fields in FIG. 3B, and select the “select device” button 324. Relevant data for the selected device will then be shown (e.g., a warning 326a, an image 326b of the device, a name/description 326c of the device, and/or a data table 326d), as seen on the right side of FIG. 3B, with the option to select a “link to full MRI instructions” button 328 for the particular implant that is selected.

In a further variation, the software tool can be adapted with functionality for patients with multiple implants within the same anatomical region. In this instance, the software tool can be configured to allow selection of multiple implant devices and overlay (combine, merge) several different artefacts/implant models.

As shown in FIGS. 3C, 3D, and 3E, in addition to the tabs 302, 304, 306, 308 along the top of the window, these GUI screens 330, 340, 350 of the software tool can also have additional tabs or buttons along the left side of the window, including an “axial view” tab/button 392, a “coronal view” tab/button 394, a “sagittal” view tab/button 396, and a “3D view” tab/button 398, which enable the software tool to display various different views to enable the user to investigate from different corresponding angles.

FIG. 3C shows a GUI screen 330 that is displayed by the software tool when the “implant location” tab 306 is selected, according to an example embodiment. In this example, the 3D view tab/button 398 has been selected. The window shows a 3D image of the recipient's head 332, where a selected implant 335 (e.g., selected via GUI screen 320) is positioned on the side of the recipient's head 332 behind the ear. In FIG. 3C, the position of the implant shown can be determined by default (e.g., based on the particular device, device type/subtype, etc. that is selected), or can be placed manually by the user. In addition, the user can be able to adjust the position of the implant 335 in GUI screen 330. This implant location adjustment feature enables different effects of different locations with respect implant-induced image artefacts (i.e., to visualize how the size and/or shape of the artefact might change, for the better or worse, for different possible implant positions) on the GUI screen 340 described below with reference to FIG. 3D.

FIG. 3D shows a GUI screen 340 that is displayed by the software tool when the “annotated data” tab 308 is selected, according to an example embodiment. Again, the 3D view tab/button 398 has been selected in this example. The window shows an example for the selected implant 335 with a magnet 345 in place. In FIG. 3D, a (partial) pre-operative MRI image 342 with a 3D image artefact 346 overlaid thereon (covering part of the MRI image 342) is shown in the window, and corresponds to one or more steps of the workflow described above with reference to FIG. 2 (where the software tool places the 3D model of the implant on the preoperative imaging data, calculates the expected best/worst-case implant artefact volumes, and/or displays a combined dataset so that the clinician can scroll through the combined dataset to investigate the visibility of an anatomical ROI, or lack thereof, for example). In this combined image/view, the user is looking from the top down. In this specific slice/height, only the magnet 345 of the implant 335 can be seen (the implant body is lower in the body of the recipient).

FIG. 3E shows a GUI screen 350 that is displayed by the software tool when the annotated data tab 308 is selected, according to an example embodiment. The window shows an example for an implant 335 with the magnet 345 removed, in which the magnet 345 is replaced with a non-magnetic plug 355. In FIG. 3E, a (complete) pre-operative MRI image 352 (without a 3D image artefact overlaid thereon) is shown in the window, and corresponds to the last step of the workflow described above with reference to FIG. 2 (where the software tool displays a combined dataset so that the clinician can scroll through the combined dataset to investigate the visibility of an anatomical ROI, for example). For example, this enables the clinician to investigate one or more anatomical regions of interest (ROIs) that would not be visible in a future MRI scan, due to the presence of the implant 335 (and the corresponding implant-induced image artefact 346 caused by the magnet 345). In this image/view, the user is looking from the top down. In this specific slice/height, only the non-magnetic plug 355 can be seen (the implant body is lower in the body of the recipient).

In some example embodiments, the user/clinician can be able to toggle between GUI screen 340 of FIG. 3D and GUI screen 350 of FIG. 3E by clicking on the magnet 345 or the non-magnetic plug 355 to switch back and forth between these views. Additionally or alternatively, the software tool (GUI) can provide a selection box that allows selecting the images for the allowable configurations of the selected implant (e.g., implant with magnet, implant without magnet but with non-magnetic plug, implant without magnet and without non-magnetic plug, etc.). Other non-limiting examples for implementing this view changing feature include a dedicated button, menu item, or other command. This feature enables the clinician to investigate anatomical regions of the recipient that are hidden by the artefact 346, for example.

Even considering that an artefact (e.g., the implant-induced image artefact 346) can be quite large relative to the size of the recipient's head, and that there are limited locations where the magnet can be positioned, there can be a significant benefit to moving the position of the implant magnet. When the software tool overlays the expected artefact on the pre-operative MRI images of the recipient, the artefact shape can be adapted based on the implant position.

The software tool can also entail functionalities that place reasonable constraints on the placement of implants, by taking into consideration both device-related factors (e.g., electrode length, expected performance, or the presence of abandoned leads) and anatomical factors (e.g., the presence of radical cavities or scar/tumor tissue). A complementary functionality of the software tool is that it can be able to recommend an optimal implant location based on the expected device performance at that location and the expected image artefact (and the impact on the diagnostic visibility of tissue).

The software tool can also include functionalities where the imaging data is fitted with an anatomical atlas to look at tissue regions not affected by the artefact, or to identify regions that are impacted by the artefact, for example. In some example embodiments, the overlay can be a color-coded semi-transparent overlay, where the colors allow to distinguish between missing data (i.e., the large black hole/dark shadow in the image of FIG. 3D), blurring of the image (i.e., at the edges of the artefact), and geometric distortions.

The software tool can further contain a functionality that indicates when a scanner type and/or scanner setting that is not allowed, per the implantable medical device guidelines, is selected (e.g., 3T setting is selected for a 1.5T conditional device, and/or pulse sequence settings that need to be made before starting the MRI). Feedback can be provided for the available imaging datasets to indicate whether or not that specific sequence can be performed, according to the allowable conditions of use listed in the MRI safety information. For example, the feedback can be a recommendation to reduce the setting(s) (e.g., from 3T down to 1.5T setting).

Additionally, the software tool can allow objective comparison between multiple implants and/or device placements, and how these devices and/or placements impact the artefact dimensions. This functionality would help in identifying the optimal condition(s) (e.g., the best particular device and/or placement among the available options).

FIG. 4 is a flowchart of a method 400 for MRI artefact visualization using pre-operative imaging data, according to an example embodiment.

At step 410, the method 400 includes obtaining pre-operative magnetic resonance imaging (MRI) data. At step 420, the method 400 also includes obtaining a three-dimensional (3D) artefact model for an implant of a recipient based on an implant type. At step 430, the method 400 further includes displaying the pre-operative MRI data with the 3D artefact model of the implant overlaid thereon so as to visualize an implant-induced image artefact for the recipient according to a given (e.g., selected) implant location (e.g., the method includes overlaying the 3D artefact model of the implant on the pre-operative MRI data for the given implant location).

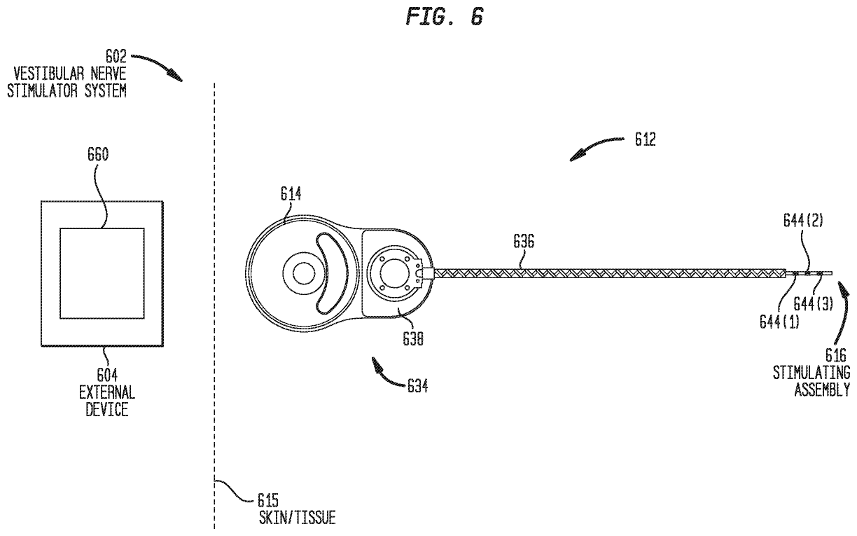

FIG. 5 is a flowchart of a method 500 for MRI artefact visualization using pre-operative imaging data, according to another example embodiment.

At step 510, the method 500 includes obtaining imaging data associated with an anatomical region of a recipient of an implantable component prior to implantation of the implantable component in the anatomical region. At step 520, the method 500 includes obtain a three-dimensional (3D) artefact model for the implantable component. At step 530, the method 500 includes determining a selected implantable location for the implantable component in the anatomical region. At step 540, method 500 includes displaying the imaging data in combination with the 3D artefact model for the selected implantable location.

One important advantage of the techniques described above with reference to FIGS. 2, 3A-3E, 4, and 5 is for the recipient and the caregivers of the recipient to be able to determine whether a head MRI is likely to be successful in visualizing a given ROI. If the techniques presented herein can eliminate MRI as a viable option for a particular recipient, then the recipient and radiology clinic will save significant effort and time.

Thus, a particular benefit for the clinician is that it becomes easier to assess the added diagnostic value of having an AIMD recipient scanned using MRI. The techniques presented herein can be implemented to advise clinicians on the effect of the implant on future MRI procedures, and what the impact of the remaining artefact would be on the diagnostic value of the images in certain anatomical regions. Simplifying expectation management will allow better decision making, doing so avoiding misdiagnosis of a post-operative pathology. The clinician could also use the solution in pre-operative counseling to evaluate different AIMDs where there will be a definite need for post-op imaging. The techniques presented herein can be configured to predict a future MRI shadow for a range of possible implant locations that can be available to the surgeon. The position of a certain AIMD can be adapted pre-operatively based on the anatomical areas of interest. Concretely, this would mean changing the magnet implantation site to preserve some anatomical areas of interest. A further benefit for the implantable medical device industry is that it becomes easier to provide customers with realistic expectations, and for objective comparisons between devices in pre-operative counseling.

Another possible use-case for the techniques described above involves providing an ‘export’ function. For example, a radio-technician can perform these techniques as an exercise when planning for an examination (or for another doctor), export the outcomes of the study, and share the outcomes with colleagues. Providing functionality to automatically generate a report that compares different configurations and the impact on the resulting image artefact would be very useful during medical discussions, and could also have value for documenting and archiving purposes.

In addition, the invention can be used in other fields that use implantable medical devices and for various applications other than hearing implants and cochlear implants, including but not limited to epilepsy-related implants, vestibular implants, and/or bone conduction hearing implants.

### Variations and Alternatives

As previously described, the technology disclosed herein can be applied in any of a variety of circumstances and with a variety of different devices. Example devices that can benefit from technology disclosed herein are described in more detail in FIG. 6. The techniques of the present disclosure can be applied to other devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 6 illustrates an example vestibular stimulator system 602, with which embodiments presented herein can be implemented. As shown, the vestibular stimulator system 602 comprises an implantable component (vestibular stimulator) 612 and an external device/component 604 (e.g., external processing device, battery charger, remote control, etc.). The external device 604 comprises a transceiver unit 660. As such, the external device 604 is configured to transfer data (and potentially power) to the vestibular stimulator 612.

The vestibular stimulator 612 comprises an implant body (main module) 634, a lead region 636, and a stimulating assembly 616, all configured to be implanted under the skin/tissue (tissue) 615 of the recipient. The implant body 634 generally comprises a hermetically-sealed housing 638 in which RF interface circuitry, one or more rechargeable batteries, one or more processors, and a stimulator unit are disposed. The implant body 634 also includes an internal/implantable coil 614 that is generally external to the housing 638, but which is connected to the transceiver via a hermetic feedthrough (not shown).

The stimulating assembly 616 comprises a plurality of electrodes 644(1)-(3) disposed in a carrier member (e.g., a flexible silicone body). In this specific example, the stimulating assembly 616 comprises three (3) stimulation electrodes, referred to as stimulation electrodes 644(1), 644(2), and 644(3). The stimulation electrodes 644(1), 644(2), and 644(3) function as an electrical interface for delivery of electrical stimulation signals to the recipient's vestibular system.

The stimulating assembly 616 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 612, the external device 604, and/or another external device, can be configured to implement the techniques presented herein. That is, the vestibular stimulator 612, possibly in combination with the external device 604 and/or another external device, can include an evoked biological response analysis system, as described elsewhere herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method comprising:
obtaining pre-operative magnetic resonance imaging (MRI) data;
obtaining a three-dimensional (3D) artefact model for an implant of a recipient based on an implant type; and
displaying the pre-operative MRI data with the 3D artefact model of the implant overlaid thereon so as to visualize an implant-induced image artefact for the recipient according to a given implant location.

2. The method of claim 1, wherein obtaining the pre-operative MRI data includes selecting the pre-operative MRI data based on user input.

3. The method of claim 2, wherein selecting the pre-operative MRI data based on user input comprises:
identifying pre-operative MRI data of a recipient of the implant if recipient data is available for the recipient; or
identifying pre-operative MRI data corresponding to a representative dataset based on one or more parameters associated with the recipient if there is no recipient data available for the recipient.

4. The method of claim 1, wherein obtaining the 3D artefact model of the implant based on the implant type includes:
selecting the implant type based on user input.

5. The method of claim 4, wherein selecting the implant type based on the user input includes one or more of:
identifying a pathology, a manufacturer of the implant, a device, a device subtype, or a combination thereof.

6. The method of claim further comprising:
determining an expected best-case artefact volume and an expected worst-case artefact volume for the implant-induced image artefact;
wherein displaying the pre-operative MRI data with the 3D artefact model of the implant overlaid thereon includes:
displaying the expected best-case artefact volume or the expected worst-case artefact volume for the implant-induced image artefact.

7. The method of claim 6, wherein the expected best-case artefact volume and the expected worst-case artefact volume for the implant-induced image artefact are provided by a manufacturer of the implant.

8. The method of claim 1, wherein displaying the pre-operative MRI data with the 3D artefact model of the implant overlaid thereon so as to visualize the implant-induced image artefact includes:
providing a display enabling a clinician to scroll through a combined dataset to investigate visibility of a particular anatomical region of interest (ROI) of the recipient.

9. The method of claim 1, wherein displaying the pre-operative MRI data with the 3D artefact model of the implant overlaid thereon so as to visualize the implant-induced image artefact includes:
providing a display indicating which anatomical parts of the recipient cannot be reliably visualized for the recipient using MRI due to the implant-induced image artefact according to the given implant location.

10. One or more non-transitory computer readable storage media comprising instructions that, when executed by a processor, cause the processor to:
obtain imaging data associated with an anatomical region of a recipient of an implantable component prior to implantation of the implantable component in the anatomical region;
obtain a three-dimensional (3D) artefact model for the implantable component;
determine a selected implantable location for the implantable component in the anatomical region; and
display the imaging data in combination with the 3D artefact model for the selected implantable location.

11. The one or more non-transitory computer readable storage media of claim 10, wherein the instructions that cause the processor to determine a selected implantable location for the implantable component in the anatomical region comprise instructions operable to:
receive a user input indicating the selected implantable location for the implantable component in the anatomical region.

12. The one or more non-transitory computer readable storage media of claim 10, wherein the instructions that cause the processor to display the imaging data in combination with the 3D artefact model for the selected implantable location comprise instructions operable to:
display the 3D artefact model of the implant overlaid on the imaging data.

13. The one or more non-transitory computer readable storage media of claim 10, wherein the instructions that cause the processor to obtain imaging data associated with the anatomical region of the recipient of comprise instructions operable to:
obtain pre-operative magnetic resonance imaging (MRI) data associated with the anatomical region of the recipient.

14. The one or more non-transitory computer readable storage media of claim 10, wherein the instructions that cause the processor to obtain imaging data associated with then anatomical region of the recipient comprise instructions operable to:
obtain the imaging data in response to a user input.

15. The one or more non-transitory computer readable storage media of claim 10, wherein the instructions cause the processor to:
determine a type of the implantable component; and
obtain the 3D artefact model of the implantable component based on the type of the implantable component.

16. (canceled)

17. The one or more non-transitory computer readable storage media of claim 10, wherein the instructions further cause the processor to:
determine an expected worst-case artefact volume for an implant-induced image artefact associated with the implantable component;
wherein the instructions operable to display the imaging data in combination with the 3D artefact model for the selected implantable location comprise instructions operable to:
displaying a 3D artefact model of the implant representing the expected worst-case artefact volume.

18. The one or more non-transitory computer readable storage media of claim 17, wherein the instructions further cause the processor to:
calculate the expected worst-case artefact volume for the implant-induced image artefact based on model information provided by a manufacturer of the implant.

19. (canceled)

20. (canceled)

21. A system, comprising:
a display screen;
a memory storing computer-readable instructions; and
at least one processor operably coupled to the display screen and the memory, wherein the at least one processor is configured to execute the computer-readable instructions to:
obtain pre-operative imaging data associated with a recipient,
obtain an artefact model associated with an implant configured to be implanted in the recipient, and
display, on the display screen, the artefact model associated with the implant overlaid on pre-operative imaging data according to a given implant location.

22. The system of claim 21, wherein to obtain the pre-operative imaging data associated with the recipient, the at least one processor is configured to obtain magnetic resonance imaging (MRI) data associated with the recipient.

23. The system of claim 21, wherein to obtain the artefact model associated with the implant configured to be implanted in the recipient, the at least one processor is configured to obtain a three-dimensional (3D) artefact model associated with the implant.

24-28. (canceled)
